(19) United States
(12) Patent Application Publication
(10) Pub. No.: US 2024/0392256 A1
(43) Pub. Date: Nov. 28, 2024

(54) Methods and Compositions for the Labeling and Selection of Antigen-Specific T-Cells

(71) Applicant: THE REGENTS OF THE UNIVERSITY OF CALIFORNIA, Oakland, CA (US)

(72) Inventors: Christopher S. Seet, Los Angeles, CA (US); Jocelyn T. Kim, Los Angeles, CA (US); Gay M. Crooks, Los Angeles, CA (US)

(73) Assignee: THE REGENTS OF THE UNIVERSITY OF CALIFORNIA, Oakland, CA (US)

(21) Appl. No.: 18/262,709

(22) PCT Filed: Jan. 25, 2022
(86) PCT No.: PCT/US2022/013650

§ 371 Date: Jul. 24, 2023

Related U.S. Application Data
(60) Provisional application No. 63/141,374, filed on Jan. 25, 2021.

(51) Int. Cl. C12N 7/00; C07K 14/005; C07K 14/74; C12N 15/86

(52) U.S. Cl. CPC C12N 7/00; C07K 14/005; C07K 14/70539; C12N 15/86; C07K 2319/03; C07K 2319/60; C12N 2740/15022; C12N 2740/15043; C12N 2740/15045

(57) ABSTRACT

The current disclosure provides for the simultaneous isolation and selection of antigen-specific T cells by providing engineered proteins, cell lines, and viruses that have a peptide-major histocompatibility complex (pMHC) on the surface of the virus that facilitates transduction of the T cell that, through the T cell receptor (TCR), specifically binds to the peptide in the pMHC complex. Accordingly, aspects of the disclosure relate to proteins, cells, and viral particles that can achieve these methods.

This application is claims benefit of priority of U.S. Provisional Application No. 63/141,374, filed Jan. 25, 2021, which is hereby incorporated by reference in its entirety.

### I. STATEMENT OF GOVERNMENT SUPPORT

This invention was made with government support under Grant Numbers AI028697, KL2TR001882, and ULITR001881, awarded by the National Institutes of Health. The government has certain rights in the invention.

### Background

### II. FIELD OF THE INVENTION

The invention generally relates to the field of molecular biology and medicine. More particularly, it concerns compositions and methods for labeling and selecting antigen-specific T cells which can then be used for downstream applications such as adoption transfer methods or identification of novel TCRs.

### III. BACKGROUND

Identification of T cells reactive to specific antigens enables the development of cellular immunotherapy by either providing primary antigen-specific T cells for adoptive transfer, or enabling the isolation of sequences for T cell receptors (TCRs) reactive to specific antigens, which may then be transferred into T cells for adoptive cell therapy. Therefore, cellular immunotherapy using either primary antigen-specific T cells, or T cells “redirected” with antigen-specific TCRs may be used to target cells expressing disease-associated antigens, and applied to the treatment of cancer, infectious diseases, or autoimmune diseases, among other conditions.

The isolation of T cells reactive to specific antigens, however, is challenging due to the rarity of endogenous T cells specific to any single antigen, and the technical challenges associated with current methods of identifying antigen-specific T cells. Examples of current methods to identify antigen-specific T cells include: 1) labeling cells with peptide-MHC multimers, which requires knowledge or prediction of antigenic peptide sequences, and an enriched source of antigen-specific T cells to increase their frequency (e.g. TILs or other antigen-primed T cell populations); this method involves costly production of recombinant soluble MHC molecules, peptide synthesis, and the technically variable UV-exchange method; 2) functional assays for T cell response to antigen presenting cells (APCs), which requires a functional T cell pool (i.e. non-exhausted), a suitable HLA-matched antigen presenting cell (e.g. autologous MoDC) and a specific activation marker or process (e.g. CD107a, 4-1BB, cytokine release, proliferation); this method is not suitable for high-throughput applications, certain T cell pools are exhausted and may not respond in functional assays, and activation markers may be upregulated non-specifically on bystander T cells; and 3) T cell/APC doublet sorting, which is experimental and not robustly validated.

Thus, there is a need in the art for improved methods for isolating antigen-specific T cells.

### SUMMARY OF INVENTION

The current disclosure provides for the simultaneous identification and selection (for example through enhanced survival or proliferation) of antigen-specific T cells by providing engineered proteins, cell lines, and viruses that have a peptide-major histocompatibility complex (pMHC) on the surface of the virus that facilitates transduction of the T cell that, through the T cell receptor (TCR), specifically binds to the pMHC complex. Accordingly, aspects of the disclosure relate to proteins, cells, and viral particles that can achieve these methods.

Aspects of the disclosure relate to a chimeric protein comprising at least a portion of a MHC polypeptide and viral protein or a fragment thereof. Also described is a chimeric protein comprising a detection gene and a viral protein or a fragment thereof. In some aspects, the viral protein comprises a viral particle protein that is selectively packaged in viral cells. Further aspects relate to a chimeric protein comprising at least a portion of a major histocompatibility complex and a transmembrane domain. In some aspects, the transmembrane domain comprises a transmembrane domain from a tetraspanin protein. In some aspects, the transmembrane domain comprises a transmembrane domain from a tetraspanin protein. Further aspects relate to a nucleic acid encoding for a chimeric protein or the disclosure. Further aspects relate to a viral vector comprising a nucleic acid of the disclosure. Further aspects of the disclosure relate to a host cell comprising a chimeric protein of the disclosure, a nucleic acid of the disclosure, and/or a viral vector of the disclosure. Also described is a viral vector comprising one or more of a MHC polypeptide, SVGmu, mutant VSV-G, and a detection gene. Further aspects relate to a host cell comprising all or a portion of a MHC polypeptide, wherein the host cell is a viral packaging cell line. Further aspects of the disclosure relate to a host cell comprising one or more nucleic acids encoding all or a portion of a MHC polypeptide, wherein the host cell is a viral packaging cell line. Further aspects relate to a method comprising incubating a host cell of the disclosure under conditions suitable for the production of viral particles and isolating viral particles. Further aspects relate to a viral particle produced by a host cell of the disclosure. Further aspects relate to a viral particle comprising all or a portion of a MHC polypeptide linked to the surface of the viral envelope. Further aspects relate to a virus or virus preparation comprising a plurality of viral particles of the disclosure. Further aspects relate to a method for labeling and/or selecting antigen-specific T cells, the method comprising infecting a population of T cells with a virus of the disclosure, wherein T cells specific for the antigenic peptide-MHC polypeptide on the surface of the virus are infected with the virus. Even further aspects relate to kits comprising one or more of the proteins, peptides, antigens, host cells, or viral particles described herein.

The disclosure also provides for a viral particle comprising a nucleic acid encoding a single chain trimer (SCT), a SVGmu protein and/or nucleic acid encoding a SVGmu protein, and a nucleic acid encoding a detection gene, wherein the viral particle comprises a nucleic acid encoding a fusion protein comprising the SCT and SVGmu. Also described is a viral particle comprising a nucleic acid encoding a single chain trimer (SCT), a SVGmu protein, and a nucleic acid encoding a detection gene, wherein the detection gene encodes for a fusion protein comprising the detection gene and a viral protein, and wherein the viral protein comprises a matrix protein (MA), nucleocapsid protein (NC), or viral protein R (VPR).

In some aspects, the disclosure relates to a virus comprising one or more viral particles of the disclosure, wherein each viral particle comprises a barcode that is unique to the peptide or pMHC expressed. In some aspects, the method further comprises contacting the viral particles with one or more peptides that bind to the MHC polypeptide.

In some aspects, the host cell comprises a viral packaging cell. In some aspects, the packaging cell comprises a 293T cell.

In some aspects of the chimeric protein, the MHC polypeptide or detection gene is at the C-terminal or N-terminal end of the viral protein or viral protein fragment. In some aspects, the viral protein or fragment comprises a protein expressed from the viral gag or env genes, or a fragment thereof. In some aspects, the viral protein or fragment comprises a viral envelope protein and wherein the envelope protein is fusogenic and T cell non-tropic.

In some aspects, the envelope protein comprises a viral glycoprotein. In some aspects, the viral protein comprises a viral matrix protein (MA), nucleocapsid protein (NC), (NC), or viral protein R (VPR). In some aspects, the viral protein comprises NC. In some aspects, the viral glycoprotein comprises SVGmu. SVGmu is further described in Yang et al., Engineered lentivector targeting of dendritic cells for in vivo immunization. Nat Biotechnol. 2008 March; 26(3): 326-34, which is herein incorporated by reference. In some aspects, the SVG comprises one or more of a deletion of amino acids 61-64, mutations of 157KR158 into 157AA158 of the SVG E2, and insertion of 10-amino acid tag sequence (MYPYDVPDYA—SEQ ID NO:1) between amino acids 71 and 74 of SVG E2. In some aspects, the viral glycoprotein comprises the binding-defective vesicular stomatitits virus envelope VSV-GS. This protein is described in Zhang et al., Cell-specific targeting of lentiviral vectors mediated by fusion proteins derived from Sindbis virus, vesicular stomatitis virus, or avian sarcoma/leukosis virus. Retrovirology. 2010 Jan. 25; 7:3. doi: 10.1186/1742-4690-7-3, which is herein incorporated by reference. In some aspects, the envelope protein comprises the vesicular stomatitis virus G protein (VSV-G) with K47Q and R354A substitutions. In some aspects, the virus is integrase deficient and canno integrate into the host genome. For example, the virus may harbor a viral integrase mutant such as a D64V substitute of the viral integrase protein.

In some aspects, the viral protein comprises an ecotropic envelope protein or a fragment thereof. In some aspects, the envelope protein comprises a class I, class II, or class III envelope protein.

In some aspects, the MHC polypeptide or fragment thereof comprises a polypeptide from a class I or class II MHC. MHC polypeptide aspects are further described herein.

In some aspects, the viral vector comprises a lentiviral, herpes simplex viral (HSV), or vaccinia viral vector. In some aspects, the viral vector comprises a retroviral or AAV viral vector. In some aspects, the MHC polypeptide is non-covalently linked to the host cell through one or more adaptor molecules. The viral vector may comprise or further comprise a MA, NC, or VPR viral gene. In some aspects, the MHC polypeptide, SVGmu, mutant VSV-G, and/or detection gene expressed from the viral vector is in a fusion protein with the viral gene.

In some aspects, the host cell further comprises an antigen or fragment thereof. In some aspects, the host cell further comprises a nucleic acid encoding an antigen or fragment thereof. In some aspects, the antigen or fragment thereof comprises a peptide. In some aspects, the peptide is 13-25 amino acids in length. In some aspects, the peptide is at least, at most, or exactly 5, 6, 7, 8, 9, 10, 11, 12, 13, 14, 15, 15, 17, 18, 19, 20, 21, 22, 23, 24, 25, 26, 27, 28, 29, 30, 31, 32, 33, 34, or 35 amino acids in length (or any range derivable therein). In some aspects, the host cell comprises a fusion protein or a nucleic acid encoding a fusion protein, wherein the fusion protein comprises the peptide, class I heavy chain, and α2-microglobulin light chain. In some aspects, the fusion protein comprises a single chain trimer (SCT). In some aspects, the fusion protein comprises a class II heavy chain and β2-microglobulin light chain. In some aspects, the fusion protein comprises a class I heavy chain and a β2-microglobulin light chain. In some aspects, the fusion protein further comprises a peptide fused to the MHC peptide and viral protein. In some aspects, the fusion protein is not further fused to a peptide. The host cell may comprise a viral envelope protein with disrupted native receptor binding that retains fusion activity. In some aspects, the host cell comprises mutant VSV-G or SVGmu.

In some aspects, the cell further comprises a nucleic acid encoding a detection gene. In some aspects, the detection gene comprises green fluorescent protein (GFP). In some aspects, the detection gene comprises a cell surface protein. In some aspects, the cell surface protein is not expressed on T cells. In some aspects, the cell further comprises a nucleic acid encoding a selective advantage gene. In some aspects, the selective advantage gene comprises a pro-survival gene, a pro-proliferative gene, or an antibiotic resistance gene, or combinations thereof. In some aspects, the detection gene and/or selective advantage gene are encoded in the viral genome on a viral vector. The detection gene may encode for a fusion protein of a detection gene and a viral protein. In some aspects, the nucleic acids comprising the detection gene and/or selective advantage gene are flanked at the 3′ and/or 5′ ends by long terminal repeats (LTRs) that facilitate the insertion of the detection and/or selective advantage genes into the genome of an infected cell. In some aspects, the host cell further comprises one or more proteins or nucleic acids that encode for proteins that facilitate viral production in the cells, wherein at least one of the proteins comprises an envelope protein that is T cell non-tropic and fusogenic. In some aspects, the host cell comprises one or more of the nucleic acids of the disclosure integrated into the genome of the host cell. In some aspects, the host cell comprises one or more of the nucleic acids of the disclosure transiently expressed in the host cell. In some aspects, the host cell comprises or further comprises a peptide library comprising a plurality of nucleic acids, wherein each nucleic acid encodes for one peptide and wherein the library encodes for 2-1000 different peptides. In some aspects, the library encodes for at least, at most, or exactly 2, 10, 50, 100, 200, 300, 400, 500, 600, 700, 800, 900, 1000, 1200, 1400, 1600, 1800, 2000, 2500, 3000, 3500, 4000, or 5000 (or any derivable range therein) peptides. In some aspects, each nucleic acid further comprises a barcode. In some aspects, the nucleic acid encoding the peptide and the nucleic acid encoding the barcode is genetically linked. In some aspects, the barcode is flanked at the 3′ and/or 5′ ends by long terminal repeats (LTRs) that facilitate the insertion of the barcode into the genome of an infected cell. In some aspects, the MHC polypeptide is not flanked at the 3′ and 5′ ends by LTRs. In some aspects, an endogenous MHC or HLA host cell gene is mutated. In some aspects, the endogenous MHC or HLA host cell gene is mutated to reduce or eliminate protein expression, protein activity, or protein cell membrane localization. In some aspects, the host cell is deficient for MHC or HLA and/or comprises a HLA gene disruption. Methods for disruption of the endogenous MHC genes are described herein.

In some aspects, the MHC polypeptide is non-covalently linked to the viral envelope through one or more adaptor molecules. For example, the viral envelope may comprise biotin binding peptide, and the viral prep may then be incubated with biotinylated pMHC molecules, which then bind to the biotin binding peptide on the surface of the virus. An adapter protein (such as a chimeric protein expressing a biotin-binding domain or other adapter) could also be expressed as a stand-alone protein not associated with the envelope protein on the virus surface, to which pMHC molecules may subsequently be attached. In some aspects, the MHC polypeptide is non-covalently linked to the envelope through biotin and a biotin-binding peptide. In some aspects, a peptide is covalently or non-covalently linked to the MHC polypeptide. The peptide may be non-covalently linked to the MHC through binding specificity or covalently linked though a peptide bond, for example. Further aspects of adaptor pairs include eMA and mSAH biotin binding monomers (as described in Situ et al, Versatile targeting system for lentiviral vectors involving biotinylated targeting molecules, Virology. 2018 December; 525:170-181, which is incorporated by reference), AviTag, a peptide allowing biotinylation by the enzyme BirA and streptavidin (GLNDIFEAQKIEWHE—SEQ ID NO:2), Calmodulin-tag, a peptide bound by the protein calmodulin (KRRWKKNFIAVSAANRFKKISSSGAL—SEQ ID NO:3), E-tag, a peptide recognized by an antibody (GAPVPYPDPLEPR—SEQ ID NO:4), FLAG-tag, a peptide recognized by an antibody (DYKDDDDK—SEQ ID NO:5), HA-tag, a peptide from hemagglutinin recognized by an antibody (YPYDVPDYA—SEQ ID NO:6). Myc-tag, a peptide derived from c-myc recognized by an antibody (EQKLISEEDL—SEQ ID NO:7), SBP-tag, a peptide which binds to streptavidin (MDEKTTGWRGGHVVEGLAGELEQLRARLEHHPQGQREP—SEQ ID NO: 8). SpyTag, a peptide which binds covalently to SpyCatcher protein (AHIVMVDAYKPTK—SEQ ID NO:9), and SnoopTag, a peptide which binds covalently to SnoopCatcher protein (KLGDIEFIKVNK—SEQ ID NO:10).

In some aspects, the MHC polypeptide is linked to the envelope through a transmembrane domain embedded within the membrane of the envelope. In some aspects, the MHC polypeptide comprises a chimeric protein comprising at least a portion of a major histocompatibility complex (MHC) polypeptide and a viral protein or a fragment thereof. In come aspects, the MHC polypeptide is at the C-terminal or N-terminal end of the viral protein or viral protein fragment. In some aspects, the viral protein or fragment comprises a viral envelope protein and wherein the envelope protein is fusogenic and non-tropic for T cells. In some aspects, the viral protein comprises a viral envelope protein and wherein the envelope protein is fusogenic and non-tropic for human T cells. In some aspects, the viral protein comprises MA, NC, or VPR.

In some aspects, the peptide is linked to the MHC polypeptide through non-covalent interactions. In some aspects, the peptide is linked to the MHC polypeptide through a covalent bond and wherein the covalent bond comprises a peptide bond. In some aspects, the peptide linked to the MHC polypeptide comprises a fusion protein comprising a single chain trimer (SCT). In some aspects, the virus further comprises a nucleic acid encoding a detection gene. In some aspects, the virus further comprises a nucleic acid encoding a selective advantage gene. In some aspects, the selective advantage gene comprises a pro-survival gene, a pro-proliferative gene, or an antibiotic resistance gene, or combinations thereof. In some aspects, the nucleic acids comprising the detection gene and/or selective advantage gene are flanked at the 3′ and/or 5′ ends by long terminal repeats (LTRs) that facilitate the insertion of the detection and/or selective advantage genes into the genome of an infected cell. In some aspects, the barcode is flanked at the 3′ and/or 5′ ends by long terminal repeats (LTRs) that facilitate the insertion of the barcode into the genome of an infected cell.

In some aspects, the T cells comprise peripheral blood mononuclear cells (PBMC) or tumor infiltrating lymphocytes (TIL). In some aspects, the methods of the disclosure further comprise selecting for the antigen-specific T cells. In some aspects, the method further comprises detecting cells expressing the detection gene and isolating and/or counting the detected cells. In some aspects, isolating and/or counting the detected cells comprises flow cytometry, magnetic sorting, microscopy, or gel electrophoresis. In some aspects, the method further comprises sequencing all or part of the genome or transcriptome of the isolated cells.

In some aspects, the kits further comprise one or more reagents for performing the methods of the disclosure, such as one or more expression constructs or viral vectors expressing one or more viral proteins, packaging cells, transduction and/or transfection reagents, or cell culture reagents.

A kit may include one or more components that are separate or together in a suitable container means, such as a sterile, non-reactive container. In some aspects, cells or viruses are provided that contain one or more nucleic acid constructs that encode the polypeptides of the disclosure. The kit may comprise nucleic acids, vectors, viral particles, host cells, or other aspects of the disclosure and may be used to perform methods of the disclosure.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one.” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z.” “(x and y) or z.” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments and aspects are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIG. 1. Is a schematic showing viral targeting based on TCR specificity to identify and expand antigen specific T cells.

FIG. 2 shows a proof of concept experiment using an engineered lentivirus produced from 293T cells expressing an HLA-A*0201/B2M/NY-ESO-1 SLLMWITQV single chain trimer (293T-1073ESO), expressing SVGmu, and containing an emerald GFP detection gene and used to transduce either unmanipulated Jurkat cells or Jurkat cells expressing the 1G4 A*0201/NY-ESO-1 (SLLMWITQV) specific TCR. Detection of transduced cells was performed by flow cytometry for emGFP expression after 4 days.

FIG. 3 shows a schematic for isolating antigen-specific T cells using barcoded lentiviruses expressing a pMHC library.

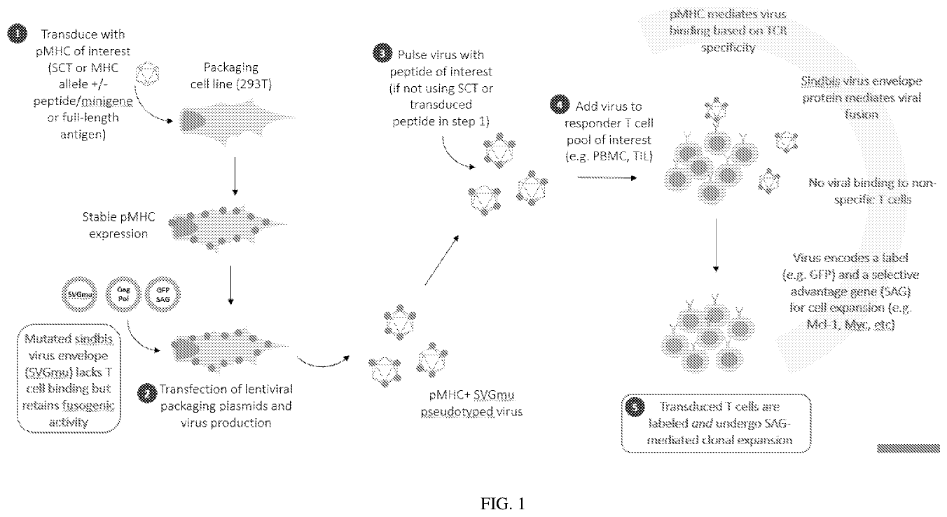

### DETAILED DESCRIPTION

### I. Definitions

As used herein, “T cell” includes all types of immune cells expressing CD3 including T-helper cells (CD4+ cells), cytotoxic T cells (CD8+ cells), T-regulatory cells (Treg) and gamma-delta T cells.

A “polypeptide” as used herein can include part of or an entire protein molecule as well as any posttranslational or other modifications.

As used in this application, the term “polynucleotide” refers to a nucleic acid molecule that either is recombinant or has been isolated free of total genomic nucleic acid. Polynucleotides may be of any length, such as 10, 50, 100, 500, 1000, 2000, 5000, 10000, or 20000 nucleotides in length, or more. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide. In this respect, the term “gene,” “polynucleotide,” or “nucleic acid” is used to refer to a nucleic acid that encodes a protein, polypeptide, or peptide (including any sequences required for proper transcription, post-translational modification, or localization).

The term “T cell non-tropic” in reference to an envelope protein refers to an envelope protein that does not bind to or facilitate infection of T cells. In reference to the whole virus, “T cell non-tropic” refers to a virus unable to bind to or infect T cells irrespective of the envelope protein's tropism. For example, a T cell non-tropic envelope protein may be one that does not confer the ability of the viral particle to infect a T cell. Such envelope proteins and viral particles may be T cell non-tropic through mutation of the envelope protein or through the use of an envelope protein that does not recognize T cells.

The term “fusogenic” refers to the process by which the virus enters a cell. Prior to entry, a virus must attach to a host cell. Attachment is achieved when specific proteins on the viral capsid or viral envelope bind to specific proteins called receptor proteins on the cell membrane of the target cell. A virus must now enter the cell, which is covered by a phospholipid bilayer, a cell's natural barrier to the outside world. The process by which this barrier is breached depends upon the virus. In enveloped viruses, the cell membrane is punctured and made to further connect with the unfolding viral envelope.

The term “single chain trimer” refers to a peptide, a β2-microglobulin, and a MHC class I heavy chain joined by linkers. MHC class II single chain trimers can be formed in an analogous fashion by linking a peptide, and MHC class II heavy and light chains.

The term “MHC polypeptide” refers to a polypeptide comprising a fragment of a MHC protein such as a polypeptide that comprises all or a fragment of a class I alpha subunit or beta-2-microglobin subunit, a class II alpha subunit, class II beta subunit, or combinations of fragments thereof. The polypeptide may have all or a part of one or more subunits that comprise the MHC, such as a polypeptide comprising a alpha1, alpha2, or alpha3 domain from a class I alpha subunit, or a alpha 1, alpha 2, beta 1, or beta 2 domain from a class II alpha or beta subunit, or combinations and fragments thereof.

### II. MHC Molecules

Aspects of the disclosure relate to viral particles and packaging cells that have at least one MHC polypeptide expressed on the surface of the viral particle or packaging cell. In some aspects, the MHC polypeptide comprises at least 2, 3, or 4 MHC polypeptides that may be expressed as separate polypeptides or as a fusion protein. Presentation of antigens to T cells is mediated by two distinct classes of molecules MHC class I (MHC-I) and MHC class II (MHC-II) (also identified as “pMHC” herein), which utilize distinct antigen processing pathways. Peptides derived from intracellular antigens are presented to CD8+ T cells by MHC class I molecules, which are expressed on virtually all cells, while extracellular antigen-derived peptides are presented to CD4+ T cells by MHC-II molecules. In certain aspects, a particular antigen is identified and presented in the antigen-MHC complex in the context of an appropriate MHC class I or II polypeptide. In certain aspects, the genetic makeup of a subject may be assessed to determine which MHC polypeptide is to be used for a particular patient and a particular set of peptides. In certain aspects, the MHC class 1 polypeptide comprises all or part of a HLA-A, HLA-B, HLA-C, HLA-E, HLA-F, HLA-G or CD-1 molecule. In aspects wherein the MHC polypeptide is a MHC class II polypeptide, the MHC class II polypeptide can comprise all or a part of a HLA-DR, HLA-DQ, or HLA-DP.

Non-classical MHC polypeptides are also contemplated for use in MHC complexes of the invention. Non-classical MHC polypeptides are non-polymorphic, conserved among species, and possess narrow, deep, hydrophobic ligand binding pockets. These binding pockets are capable of presenting glycolipids and phospholipids to Natural Killer T (NKT) cells or certain subsets of CD8+ T-cells such as Qa1, HLA-E-restricted CD8+ T-cells, or MAIT cells. NKT cells represent a unique lymphocyte population that co-express NK cell markers and a semi-invariant T cell receptor (TCR). They are implicated in the regulation of immune responses associated with a broad range of diseases.

In some aspects, all or part of a MHC polypeptide, such as a MHC polypeptide comprising the endogenous transmembrane domain or a MHC molecule lacking the endogenous transmembrane domain, may further comprise a heterologous transmembrane domain. A transmembrane moiety (or transmembrane domain) is a plurality of amino acids that is capable of extending across a biological membrane of a cell, such as the plasma membrane or a membrane of an organelle. A transmembrane moiety is a polypeptide capable of inserting into and remaining anchored within the viral surface, such as the surface of the envelope and/or the plasma membrane of a mammalian cell. When included in the fusion protein of the invention, the transmembrane moiety is capable of localizing the fusion protein to the viral surface and/or to the plasma membrane of a mammalian cell. The transmembrane moiety is a viral transmembrane moiety, such as a lentiviral transmembrane moiety. Lentiviral transmembrane moieties include for example envelope polypeptide from HIV, SIV, CAEV, BIV, BLV, and FIV or a functional fragments thereof.

The HIV-1 gp160 envelope polypeptide is a polypeptide of about 856 amino acids (See GenBank Accession numbers AA040783, AAA76671, BAA12995, and P03377, which are herein incorporated by reference) that is proteolytically processed into a gp120 and a gp41 polypeptide. The gp41 polypeptide is represented by GenBank Accession No.: NP-057856, which is herein incorporated by reference. Additional, lentiviral envelope proteins are provided by GenBank Accession numbers AAT41729 from SIV, P31627 from CAEV, P19557 from BIV, P03380 from BLV, and NP-040976 envelope from FIV.

Alternatively, the transmembrane moiety is a mammalian transmembrane moiety, such as CD28, CD8, CD9, CD63, transpanin, or IgM transmembrane moiety or functional fragment.

As used herein, a “functional fragment” of a transmembrane moiety includes any portion or fragment of a transmembrane moiety that is sufficient to localize the fusion protein to the surface of the viral particle and/or to the mammalian cell plasma membrane.

Generally, transmembrane moieties have positively charged amino acids (such as lysine and arginine) at their ends and uncharged amino acids in the center. Transmembrane proteins are capable of having an N-terminal signal sequence that targets these polypeptides to the endoplasmic reticulum. Transmembrane moieties include polypeptide sequences having one or more alpha-helical structures. Usually, an alpha-helical transmembrane domain of 25 or more amino acids in length is sufficient to span the plasma membrane. Alternatively, the transmembrane moiety may include a polypeptide having one or more beta-barrel structures. An exemplary transmembrane moiety includes the transmembrane domain from gylcophorin A (VQLAHHFSEPEITLIIFGVMAGVIGTILLISYGI; SEQ ID NO: 11). In some aspects, transmembrane domains include polypeptide sequences having two or more β-sheets.

Transmembrane moieties include polypeptides provided in the TMbase of transmembrane proteins and their membrane-spanning domains. (See, K. Hofmann & W. Stoffel (1993), TMbase-A database of membrane spanning proteins segments. Biol. Chem. Hoppe-Seyler 374,166.) The TMbase database contains information from SwissProt and other database sources. Transmembrane moieties of the present invention include polypeptides identified using the TMpred program, which makes a prediction of membrane-spanning regions and their orientation using an algorithm based on the statistical analysis of the TMbase database of naturally occurring transmembrane proteins. Other programs capable of determining transmembrane domains in a polypeptide include GREASE, TMHMM, and TMAP.

### III. Antigen and Peptide Components

Aspects of the disclosure relate to the delivery of whole antigen or peptide fragments thereof either as a nucleic acid that is expressed in the cells and presented at the surface of a packaging cell, along with a MHC molecule that is later incorporated into a virus or is expressed in the virus/packaging cell and presented at the surface of the viral particle. In some aspects, the peptide may be pulsed to prepared viral preparations comprising a MHC at the surface. In some aspects, the methods comprises pulsing a pool of peptides to a viral preparation. For example, a pool of peptides comprising multiple copies, such as at least or at most 10, 50, 100, 125, 150, 200, 300, 400, or 500 (or any derivable range therein) copies of at least or at most 2, 3, 4, 5, 6, 7, 8, 9, 10, 15, 20, 25, 30, 35, 40, 45, 50, 60, 70, 80, 90, or 100 different peptides (or any derivable range therein) may be pulsed to a viral preparation. Multiple pools of peptides may be pulsed to multiple viral preparations. The peptides from the pools may be from one antigen or from different antigens. The peptides may be known to have antigenic properties or the peptides may not be known to have antigenic properties. Aspects of the disclosure relate to the isolation of identification of novel TCRs that bind to peptides whose ability to function as an antigenic determinant may be known or unknown.

In some aspects, the peptide or antigen is provided as a nucleic acid to the packaging cells. In some aspects, the peptide is encoded on an expression vector. In some aspects, the peptide is encoded on a plasmid, vector, minigene, or other suitable vehicle for the expression of a peptide in packaging and/or viral cells. The expression vector may comprise one or more control sequences capable of enhancing, increasing, attenuating, suppressing, or inhibiting, the expression of the peptide. In some aspects, the expression vehicle, such as the plasmid, minigene, expression vector, etc. . . . may further comprise a barcode that is genetically linked to the nucleic acid encoding the peptide. In some aspects, the barcode is encoded within the viral genome and/or with elements that facilitate incorporation or integration of the barcode into the genome of the infected or transduced cell. In some aspects, the nucleic acid encoding the peptide is not integrated into the genome of the infected or transduced cell.

The expression vector may include other sequences. Expression vectors may comprise inducible or cell-type-specific promoters, enhancers or repressors, introns, polyadenylation signals, selectable markers, polylinkers, site-specific recombination sequences, and other features to improve functionality, convenience of use, and control over mRNA and/or protein expression levels. A signal sequence may direct the secretion of a polypeptide fused thereto from a cell expressing the protein. In the expression vector, nucleic acid encoding a signal sequence may be linked to a polypeptide coding sequence so as to preserve the reading frame of the polypeptide coding sequence.

### IV. Detection Gene and Selective Advantage Gene

### A. Detection Gene

In some aspects, the methods, viral particles, and packaging cells include a detection gene. Detection genes include fluorescent proteins, cell surface proteins or fragments thereof, peptide epitope tags, enzymes, enzyme substrates, enzyme cofactors, enzyme inhibitors, ligands (e.g., biotin, streptavidin or haptens) and the like. Particular examples of labels are, but not limited to, green fluorescent protein derivatives, human CD34, human CD19, horseradish peroxidase (HRP), fluorescein, FITC, rhodamine, dansyl, umbelliferone, dimethyl acridinium ester (DMAE), Texas red, luminol, NADPH and α- or β-galactosidase. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

In some aspects, the detection gene is one that confers a phenotype that allows for the discrimination of transduced cells based on fluorescence, such as GFP. In certain aspects, the detection gene comprises a fluorescent marker, an enzymatic marker, a luminescent marker, a photoactivatable marker, a photoconvertible marker, or a colorimetric marker. Flouorescent markers include, for example, GFP and variants such as YFP, RFP etc., and other fluorescent proteins such as DsRed, mPlum, mCherry, YPet, Emerald, CyPet, T-Sapphire, and Venus. Photoactivatable markers include, for example, KFP, PA-mRFP, and Dronpa. Photoconvertible markers include, for example, mEosFP, KikGR, and PS-CFP2. Luminescent proteins include, for example, Neptune, FP595, and phialidin.

### B. Selective Advantage Gene

In certain aspects, the packaging cells and viral particles include a selective advantage gene, which can be defined as a gene that confers a selective advantage to the cell that it is expressed in. The selective advantage may be dependent on or independent of a selection agent. For example, the selective advantage gene may be a drug resistance marker or an antibiotic resistance gene/marker. Examples include genes that confer resistance to neomycin, puromycin, hygromycin, DHFR, GPT, zeocin, G418, phleomycin, blasticidin, and histidinol.

In some aspects, the selective advantage gene confers a selective advantage in absence of a selection agent, such as pro-survival genes or pro-proliferative genes. Examples include MCL-1, Bcl-2, myc, myrAkt, RAS, and LMO2.

### V. Polypeptides

In some aspects, the nucleic acids, polypeptides, or proteins of the disclosure may have one or more conservative or non-conservative substitutions. Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Aspects of the disclosure include a peptide/polypeptide that is at least, at most, or exactly 75, 76, 77, 78, 79, 80, 81, 82, 83, 84, 85, 86, 87, 88, 89, 90, 91, 92, 93, 94, 95, 96, 97, 98, 99, or 100% identical (or any derivable range therein) to a peptide or polypeptide/polypeptide that has at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein to a peptide/polypeptide that starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, or 450 of any polypeptide or protein described herein.

The polypeptides, peptides, and proteins described herein may include 1, 2, 3, 4, 5, 6, 7, 8, 9, 10, 11, 12, 13, 14, 15, or more (or any derivable range therein) variant amino acids within at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of a polypeptide, peptides, or protein of the disclosure.

A polypeptide segment or fragment or a peptide as described herein may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of an antigen, peptide, protein, or polypeptide of the disclosure.

The polypeptides, proteins, antigens, or peptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

A linker sequence may be included in the peptide construction. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate nucleic acids of the disclosure, such as nucleic acid encoding a peptide and a MHC (eg, single chain trimer). In some aspects, the linker comprises a glycine serine linker. In some aspects, the linker comprises (GSGG—SEQ ID NO:12)x, wherein x=1-6. In some aspects, x=2. In some aspects, x=1, 2, 3, 4, 5, or 6 (or any derivable range therein) In some aspects, the linker comprises GSGGGSGG (SEQ ID NO:13).

### VI. Nucleic Acids

In certain aspects, the current disclosure concerns polynucleotides encoding the proteins, polypeptides, chimeric proteins, and peptides of the disclosure.

As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges there between, of a polynucleotide encoding one or more proteins or polypeptides referenced herein. It is also contemplated that a particular polypeptide may be encoded by nucleic acids containing variations having slightly different nucleic acid sequences but, nonetheless, encode the same or substantially similar protein.

In particular aspects, the invention concerns isolated nucleic acid segments and recombinant vectors incorporating nucleic acid sequences that encode a polypeptide, protein, antigen, or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other aspects, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed herein, a tag, detection gene, or other heterologous polypeptide may be added to the modified polypeptide-encoding sequence, wherein “heterologous” refers to a polypeptide that is not the same as the modified polypeptide.

In certain aspects, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNA, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference).

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked.” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain aspects may direct high level expression of the introduced DNA segment under specified conditions, such as large-scale production of recombinant proteins or peptides.

Various elements/promoters may be employed in the context of the present invention to regulate the expression of a gene. Examples of such inducible elements, which are regions of a nucleic acid sequence that can be activated in response to a specific stimulus, include but are not limited to Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990), Immunoglobulin Light Chain (Queen et al., 1983; Picard et al., 1984), T Cell Receptor (Luria et al., 1987; Winoto et al., 1989; Redondo et al.; 1990), HLA DQ and/or DQ; Sullivan et al., 1987), □ Interferon (Goodbourn et al., 1986; Fujita et al., 1987; Goodbourn et al., 1988), Interleukin-2 (Greene et al., 1989), Interleukin-2 Receptor (Greene et al., 1989; Lin et al., 1990), MHC Class II 5 (Koch et al., 1989), MHC Class II HLA-DR; Sherman et al., 1989), Actin (Kawamoto et al., 1988; Ng et al.; 1989), Muscle Creatine Kinase (MCK) (Jaynes et al., 1988; Horlick et al., 1989; Johnson et al., 1989), Prealbumin (Transthyretin) (Costa et al., 1988), Elastase I (Ornitz et al., 1987), Metallothionein (MTII) (Karin et al., 1987; Culotta et al., 1989), Collagenase (Pinkert et al., 1987; Angel et al., 1987), Albumin (Pinkert et al., 1987; Tronche et al., 1989, 1990), Fetoprotein (Godbout et al., 1988; Campere et al., 1989), γ-Globin (Bodine et al., 1987; Perez-Stable et al., 1990), □-Globin (Trudel et al., 1987), c-fos (Cohen et al., 1987), c-Ha-Ras (Triesman, 1986; Deschamps et al., 1985), Insulin (Edlund et al., 1985), Neural Cell Adhesion Molecule (NCAM) (Hirsh et al., 1990), 1-Antitrypain (Latimer et al., 1990), H2B (TH2B) Histone (Hwang et al., 1990), Mouse and/or Type I Collagen (Ripe et al., 1989), Glucose-Regulated Proteins (GRP94 and GRP78) (Chang et al., 1989), Rat Growth Hormone (Larsen et al., 1986), Human Serum Amyloid A (SAA) (Edbrooke et al., 1989), Troponin I (TN I) (Yutzey et al., 1989), Platelet-Derived Growth Factor (PDGF) (Pech et al., 1989), Duchenne Muscular Dystrophy (Klamut et al., 1990), SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988), Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell et al., 1988), Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thicsen et al., 1988; Celander et al., 1988; Choi et al., 1988; Reisman et al., 1989), Papilloma Virus (Campo et al., 1983; Lusky et al., 1983; Spandidos and Wilkie, 1983; Spalholz et al., 1985; Lusky et al., 1986; Cripe et al., 1987; Gloss et al., 1987; Hirochika et al., 1987; Stephens et al., 1987), Hepatitis B Virus (Bulla et al., 1986; Jameel et al., 1986; Shaul et al., 1987; Spandau et al., 1988; Vannice et al., 1988), Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989), Cytomegalovirus (CMV) IE (Weber et al., 1984; Boshart et al., 1985; Foccking et al., 1986), Gibbon Ape Leukemia Virus (Holbrook et al., 1987; Quinn et al., 1989).

Inducible elements include, but are not limited to MT II-Phorbol Ester (TFA)/Heavy metals (Palmiter et al., 1982; Haslinger et al., 1985; Scarle et al., 1985; Stuart et al., 1985; Imagawa et al., 1987, Karin et al., 1987; Angel et al., 1987b; McNcall et al., 1989); MMTV (mouse mammary tumor virus)—Glucocorticoids (Huang et al., 1981; Lec et al., 1981; Majors et al., 1983; Chandler et al., 1983; Lec et al., 1984; Ponta et al., 1985; Sakai et al., 1988); Interferon—poly(rl) x/poly(rc) (Tavernier et al., 1983); Adenovirus 5 E2—ElA (Imperiale et al., 1984); Collagenase—Phorbol Ester (TPA) (Angel et al., 1987a); Stromelysin—Phorbol Ester (TPA) (Angel et al., 1987b); SV40—Phorbol Ester (TPA) (Angel et al., 1987b); Murine MX Gene—Interferon, Newcastle Disease Virus (Hug et al., 1988); GRP78 Gene—A23187 (Resendez et al., 1988); B-2-Macroglobulin—IL-6 (Kunz et al., 1989); Vimentin—Serum (Rittling et al., 1989); MHC Class I Gene H-2b—Interferon (Blanar et al., 1989); HSP70—ElA/SV40 Large T Antigen (Taylor et al., 1989, 1990a, 1990b); Proliferin—Phorbol Ester/TPA (Mordacq et al., 1989); Tumor Necrosis Factor—PMA (Hensel et al., 1989); and Thyroid Stimulating Hormone Gene—Thyroid Hormone (Chatterjee et al., 1989).

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain aspects of the disclosure, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference). In some aspects, a 2A peptide is used to create multigene or polyscistronic messages.

### D. Selectable and Screenable Markers

In certain aspects of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. As an alternative, 2A peptides could be used to introduce ribosomal skips to enable expression of multiple polypeptidic or protein sequences.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available. In some aspects, the expression system comprises a second generation lentiviral packaging system in 293T cells. In some aspects, the gag, pol, and rev are on the same plasmid.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENER'S COMPLETE CONTROL. Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### VII. Viral Particles and Methods of Producing Viral Particles

### A. Enveloped Virus

The disclosure provides for viral particles and preparations comprising all or a portion of a MHC polypeptide linked to the surface of the viral envelope. Aspects of the disclosure relate to virus that are enveloped viruses. Enveloped viruses express envelope glycoproteins that enable the infection of host cells by mediating fusion between the viral envelope and host cell membranes. For many enveloped viruses, the endoplasmic reticulum (ER) of the host cell is used to support viral entry, replication, and/or assembly. Exemplary envelope virus classes include measles viruses, herpes viruses, lentiviruses, alpha viruses, pox viruses, and vaccinia viruses. Exemplary viruses include, for example, the herpes simplex virus, Newcastle-disease virus and vesicular stomatitis virus.

The engineered viruses of the disclosure may comprise viruses that do not normally cause disease or other negative effects in the infected host or cell. Alternatively, the virus may be an attenuated virus, i.e. a virus which has mutated or which has been engineered or otherwise treated such that it does not support pathogenic infection of healthy or nontarget cells.

In some aspects, the virus can be derived from the Lentivirus genus. In some aspects, the recombinant retrovirus can be derived from HIV, SIV, or FIV. In further aspects, the recombinant retrovirus can be derived from the human immunodeficiency virus (HIV) in the Lentivirus genus. Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. In vivo, HIV can infect terminally differentiated cells that rarely divide, such as lymphocytes and macrophages.

In the methods and compositions provided herein, the recombinant retroviral genomes, in non-limiting illustrative examples, lenti viral genomes, have a limitation to the number of polynucleotides that can be packaged into the viral particle. In some aspects provided herein, the polypeptides encoded by the polynucleotide encoding region can be truncations or other deletions that retain a functional activity such that the polynucleotide encoding region is encoded by less nucleotides than the polynucleotide encoding region for the wild-type polypeptide. In some aspects, the polypeptides encoded by the polynucleotide encoding region can be fusion polypeptides that can be expressed from one promoter. In some aspects, the fusion polypeptide can have a cleavage signal to generate two or more functional polypeptides from one fusion polypeptide and one promoter. Furthermore, some functions that are not required after initial ex vivo transduction are not included in the retroviral genome, but rather are present on the surface of the vims or retrovirus via the packaging cell membrane. These various strategies are used herein to maximize the functional elements that packaged within the retrovirus.

In some aspects, the recombinant retroviral genome to be packaged can be between 1,000, 2,000, 3,000, 4,000, 5,000, 6,000, 7,000, and 8,000 nucleotides on the low end of the range and 2,000, 3,000, 4,000, 5,000, 6,000, 7,000, 8,000, 9,000, 10,000, and 11,000 nucleotides on the high end of the range. Functions discussed elsewhere herein that can be packaged include required retroviral sequences for retroviral assembly and packaging, such as a retroviral env, gag, and pol coding regions, as well as a 5′ LTR and a 3′ LTR, or an active truncated fragment thereof.

### B. Packaging Cells and Systems

In one aspect, provided herein are packaging cells or systems comprising the chimeric proteins, MHC proteins, polypeptides, single chain trimers, detection gene, selective advantage gene, and/or viral support elements as described herein.

The packaging cells may be any cell suitable for virus production. In some aspects, the packaging cells is a mammalian cell that is used to make virus. Any of a wide variety of cells can be selected for in vitro production of a virus. Eukaryotic cells arc typically used, particularly mammalian cells including human, simian, canine, feline, equine and rodent cells. In illustrative examples, the cells are human cells. In further illustrative aspects, the cells reproduce indefinitely, and are therefore immortal. Examples of cells that can be advantageously used in the present invention include NIH 3T3 cells, COS cells, Madin-Darby canine kidney cells, human embryonic 293T cells and any cells derived from such cells. Highly transfectable cells, such as human embryonic kidney 293T cells, can be used. By “highly transfectable” it is meant that at least about 50%, more preferably at least about 70% and most preferably at least about 80% of the cells can express the genes of the introduced DNA.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCLIO), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, Hut-78, Jurkat, HL-60, NK cell lines (e.g., NKL, NK92, and YTS), and the like.

In any of the aspects disclosed herein, the methods of making virus can include growing a mammalian packaging cells to 50%, 60%, 70%, 80%, 90% or 95% confluence or confluence to 25%, 30%, 40%, 50%, 60%, 70%, 80%, 90% or 95% peak cell density and then splitting or diluting the cells. In some aspects, a stirred tank reactor can be used to grow the cells. In some aspects, the cells can be split at least about 1:2, 1:3, 1:4, 1:5, 1:6, 1:7, 1:8, 1:9, 1:10, 1:12, 1:15, or 1:20 using methods a skilled artisan will understand. In some aspects, the cells can be diluted to 25%, 30%, 40%, 50%, 60%, 70%, 80%, 90% or 95% peak cell density.

In some aspects, the endogenous MHC of the packaging cells has been disrupted. Methods for disrupting gene expression are known in the art and include disruption of endogenous B2M or HLA genes, or class II regulators such as CIITA. For example, the gene may be disrupted by methods known in the art, such as CRISPR/Cas9 based methods, TALENs, or site-specific genomic modifications by zinc finger nucleases.

### VIII. Methods

The current proteins, peptides, packaging cells, and methods can be useful for selectively labeling and selecting, increasing the proliferation of, and/or expanding antigen-specific T cells. The packaging cells of the disclosure are useful for making virus that, through the pMHC on the surface of the virus, infects T cells that have a T cell Receptor (TCR) that binds to and has affinity for the peptide-MHC on the surface of the virus. Since the virus is T cell non-tropic and retains its fusogenic properties, the crosslinking of the pMHC with the TCR provides for binding to the cell and the envelope protein facilitates fusion to and transduction of the T cell. Once the T cell has been transduced by the virus, the selective advantage gene and/or detection gene can be expressed in the T cell, either by integration into the host genome or by transient expression. In some aspects, the selective advantage gene and/or detection gene integrates into the genome of the T cell after transduction by the virus.

This method can be used to identify novel TCRs and/or identify peptide antigenic determinants. The peptide may be provided to the host cell as a full or partial length protein that relies on the cells endogenous peptide processing mechanisms to then produce peptide that may be displayed as pMHC on the surface of the packaging cell (and thus virus), or the peptide may be provided as a peptide library in plasmid format. In some aspects, a peptide barcode system may be provided in which a barcode is genetically linked to a peptide in a peptide library. The barcode may be encoded in the viral genome so that it is transferred into the transduced T cell and, in some aspects, inserted into the genome of the T cell. Once the T cell has been isolated and/or selected, sequencing of the T cell genomic DNA may provide the identity of the peptide on the surface of the viral particle that transduced the T cell.

Once the T cells have been isolated and/or selected, various sequencing techniques may be employed to identify TCR sequences or other elements, such as a barcode. Sequencing methods include, for example, massively parallel signature sequencing (MPSS), polony sequencing, drop-seq, cell-seq, 454 pyrosequencing, Illumina (Solexa) sequencing, SOLID sequencing, Ion Torrent semiconductor sequencing, DNA nanoball sequencing, heliscope single molecule sequencing, and single molecule real time (SMRT) sequencing.

The isolation of antigen-specific T cells and subsequent sequencing and identification of novel TCRs is useful for many downstream applications. For example, the hypervariable regions of the TCRs can be determined and used for the construction of immunotherapeutics such as native, codon-optimized, or affinity enhanced TCRs, chimeric antigen receptors, monobodies, or multi-specific molecules based on the hypervariable regions, such as the CDRs and/or based on the TCRbeta and/or TCRalpha variable regions of the TCRs. In further therapeutic applications, the isolated T cells may also be used in immunotherapeutics directly, such as in adoptive T cell experiments or therapies. In some aspects, the T cells do not have a selective advantage gene.

The current disclosure provides methods for labeling (and/or expanding) antigen-specific T cells in a single step using engineered lentiviruses to target T cells based on their TCR specificity. Aspects of the disclosure relate to an engineered virus (eg. lentivirus or retrovius) that is targeted to T cells with specific TCR specificities by virus surface expression of a specific peptide-MHC complex (pMHC) antigen (introduced into virus packaging cells prior to viral particle formation.

Endogenous viral T cell tropism is abolished by using either: 1) an envelope protein mutated to disable receptor binding while retaining fusogenic activity (examples include previously published binding-defective sindbis virus envelope SVGmu, or binding-defective vesicular stomatitits virus envelope VSV-GS); or 2) an envelope protein with non-T cell tropism.

The lentivirus will bind to antigen-specific T cells via the pMHC expressed on the surface of viral particles interacting with a TCR capable of binding this pMHC; this interaction will trigger receptor-mediated endocytosis of the viral particle, and the envelope protein will mediate virus fusion and infection of the target cell. Infected T cells with be transduced with a 1) marker for sorting transduced cells (e.g. GFP), and 2) optionally, a selective advantage gene/s (SAG) to facilitate direct clonal expansion of transduced cells. Examples: pro-survival genes (e.g. MCL-1, Bcl-2), pro-proliferative genes (e.g. Myc, myrAkt, Ras, LMO2), ab-resistance genes (e.g. neo/puro resistance), or combinations of such genes.

Antigen-specific T cells may thus be labeled and enriched in one step, and can be sorted based on transduced marker expression for single-cell TCR-sequencing. Cells transduced with a marker only (i.e. without a SAG) can be isolated and used for downstream functional applications, including adoptive transfer.

It is contemplated that efficiency and versatility will be improved by enhancing viral pMHC display: pMHC antigens can expressed in 293T cells in the single-chain trimer (SCT) format, or as native MHC. SCT or native MHC coding sequences can be stably transduced into virus packaging cells or transiently transfected during virus packaging. If using native MHC, peptide or full-length protein (antigen) coding sequences may be transduced or transfected into packaging cells, or may be added exogenously to the viral prep (i.e. peptide pulsing) after virus packaging and before exposure to T cells.

Envelope proteins may also be engineered directly with pMHC or MHC domains (i.e. MHC/envelope fusion proteins) which may enhance viral fusion and transduction over uncoupled envelope and pMHC expression. Other similar strategies include the use of pMHC/gag fusion proteins or pMHC/tetraspanin fusions to enrich for packaging and surface expression on virus particles.

Envelope proteins may also be engineered with an adapter domain (e.g biotin-binding domain) which may allow the modular use of commercially available biotinylated pMHC monomers added to the viral preparations after production. This would allow increased modularity with respect to virus MHC expression, and also would not require MHC expression in packaging cells at the time of virus production. An adapter protein (such as a chimeric protein expressing a biotin-binding domain or other adapter) could also be expressed as a stand-alone protein on the virus surface, to which pMHC molecules could later be attached.

To enhance specificity of virus binding by abolishing non-target MHC on virus particles, endogenous MHC expression on virus packaging cells may need to be ablated (e.g. by gene editing) to prevent viral presentation of endogenous, non-target MHC molecules and virus binding to alloreactive T cells.

Strategies for highly parallel interrogation of T cells reactive to multiple pMHCs: Targeting T cells in an epitope-agnostic fashion may be achieved by expressing full-length antigen and MHC of interest in the packaging cell line before virus production, as described above, relying on endogenous peptide processing and MHC-loading by packaging cells, which are then carried over to the surface of viral particles. An alternative strategy would be to express libraries of peptides in packaging cells by transfection (e.g. minigene constructs); or pulsing MHC-viral preps with peptide pools, all as described above. Alternatively, packaging cells may be transfected with a barcoded peptide or cDNA library, and the identity of the peptide-MHC could be recovered from the transduced antigen-specific T cells by deep sequencing of viral integrations for the antigen-specific barcode

### IX. Examples

The following examples are included to demonstrate preferred aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1—A Method for Identifying T Cells with Specific Antigen Reactivity

The inventors tested proof-of-concept that lentivirus particles could target T cells based on interaction between a virus-expressed pMHC and a T-cell expressed TCR. To do this they created a pMHC-expressing packaging cell line. 293T cells were transduced with a lentivirus encoding a HLA-A*0201/B2M/NY-ESO-1157-165 single chain trimer (SCT) and a mStrawberry marker. mStrawberry+ cells were FACS sorted and expanded as a stably-transduced line (293T-1073ESO, hereafter). 293T-1073ESO cells were transfected using Mirus TransIT-293 reagent with the packaging plasmids pCMV-deltaR8.9 (gag/pol-encoding), a plasmid encoding SVGmu, and the lentiviral vector plasmid pCCL-MNDU3-emGFP, encoding emerald GFP driven by the MNDU promoter. Lentiviral supernatants were collected and concentrated by ultrafiltration.

A serial titration of concentrated lentivirus was added to target cells. Jurkat cells were used as a negative control, and Jurkat cells stably transduced with the 1G4 A*0201/NY-ESO-1157-165 specific TCR were used as the TCR-expressing positive control. Target cells were analyzed for emGFP expression 4 days after exposure to virus.

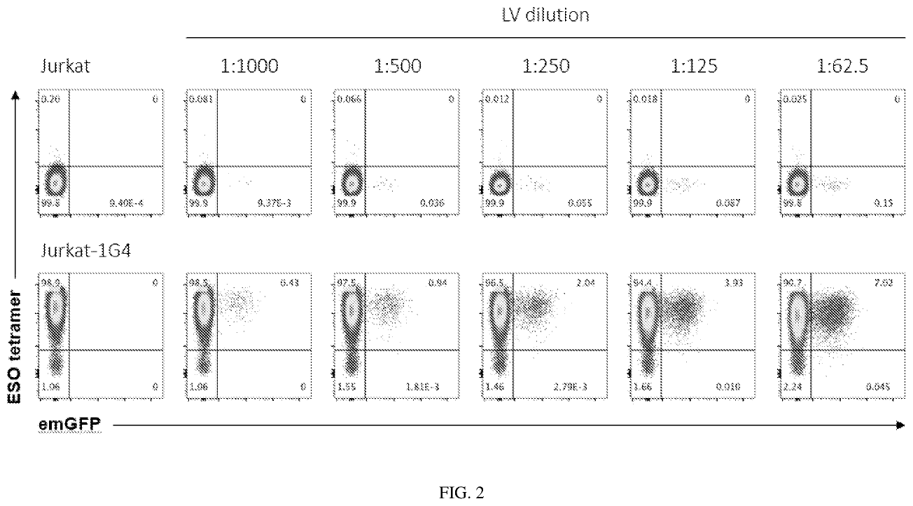

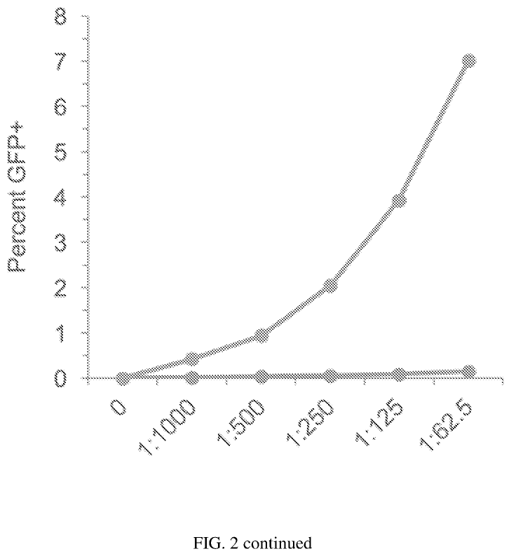

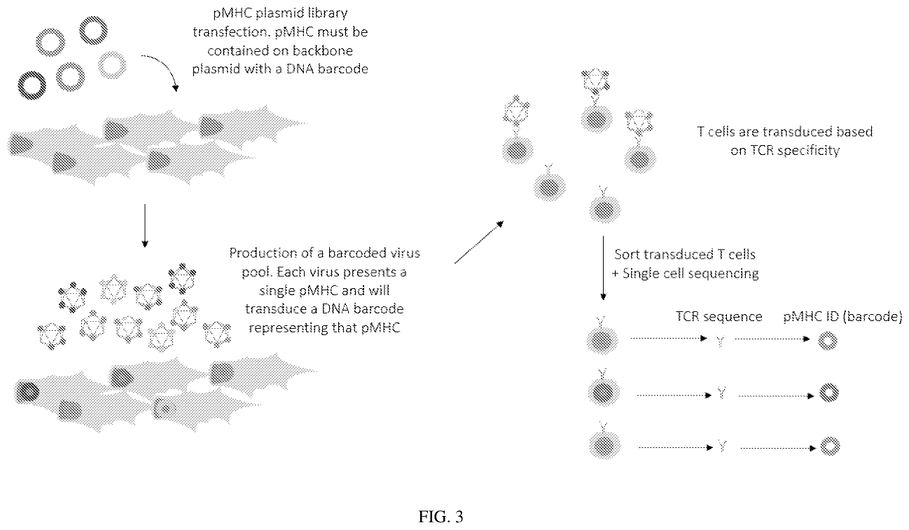

Results (FIG. 2) showed selective infection of Jurkat-1G4 cells (˜7% emGFP+ at highest viral titer) compared with control Jurkat cells (0.15% infectivity). Expression of TCR by the Jurkat-1G4 cells was confirmed by simultaneous staining with a A*0201/NY-ESO-1₁₅₇₋₁₆₅ tetramer. These data support the ability of passively packaged pMHC SCT on viral particles to mediate specific TCR-targeted binding and transduction of Jurkat cells when used in conjunction with a non-T cell tropic envelope protein and a fluorescent marker.

Although certain embodiments and aspects have been described above with a certain degree of particularity, or with reference to one or more individual embodiments or aspects, those skilled in the art could make numerous alterations to the disclosed embodiments or aspects without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or aspect or may relate to several embodiments or aspects. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

1. A viral particle comprising a nucleic acid encoding a single chain trimer (SCT), a SVGmu protein and/or nucleic acid encoding a SVGmu protein, and a nucleic acid encoding a detection gene, wherein the viral particle comprises a nucleic acid encoding a fusion protein comprising the SCT and SVGmu.

2. A viral particle comprising a nucleic acid encoding a single chain trimer (SCT), a SVGmu protein, and a nucleic acid encoding a detection gene, wherein the detection gene encodes for a fusion protein comprising the detection gene and a viral protein, and wherein the viral protein comprises a matrix protein (MA), nucleocapsid protein (NC), or viral protein R (VPR).

3. A chimeric protein comprising at least a portion of a major histocompatibility complex (MHC) polypeptide and a viral protein or a fragment thereof.

4. A chimeric protein comprising a detection gene and a viral protein or a fragment thereof.

5. The chimeric protein of claim 3 or 4, wherein the MHC polypeptide or detection gene is at the C-terminal or N-terminal end of the viral protein or viral protein fragment.

6. The chimeric protein of any one of claims 3-5, wherein the viral protein or fragment comprises a protein expressed from the viral gag or env genes, or a fragment thereof.

7. The chimeric protein of any one of claims 3-6, wherein the viral protein or fragment comprises a viral envelope protein and wherein the envelope protein is fusogenic and non-tropic for T cells.

8. The chimeric protein of claim 7, wherein the viral protein comprises a viral envelope protein and wherein the envelope protein is fusogenic and non-tropic for human T cells.

9. The chimeric protein of claim 7, wherein the envelope protein comprises a viral glycoprotein.

10. The chimeric protein of claim 9, wherein the viral glycoprotein comprises SVGmu.

11. The chimeric protein of any one of claims 3-10, wherein the viral protein comprises an ecotropic envelope protein or a fragment thereof.

12. The chimeric protein of any one of claims 3-8, wherein the viral protein comprises a matrix protein (MA), nucleocapsid protein (NC), or viral protein R (VPR).

13. The chimeric protein of claim 12, wherein the viral protein comprises a NC protein.

14. The chimeric protein of any one of claims 3-13, wherein the chimeric protein comprises a single chain HLA peptide comprising a peptide, beta-2-microglobulin (B2M), and a MHC polypeptide.

15. The chimeric protein of any one of claims 3-13, wherein the viral protein comprises a transmembrane region.

16. A chimeric protein comprising at least a portion of a major histocompatibility complex and a transmembrane domain.

17. The chimeric protein of claim 16, wherein the transmembrane domain comprises a transmembrane domain from a tetraspanin protein.

18. The chimeric protein of any one of claims 3-17, wherein the MHC polypeptide comprises a polypeptide from a class I or class II MHC.

19. A nucleic acid encoding for a chimeric protein according to any one of claims 3-18.

20. A viral vector comprising the nucleic acid of claim 19.

21. A viral vector comprising one or more of a MHC polypeptide, SVGmu, mutant VSV-G, and a detection gene.

22. The viral vector of claim 21, wherein the viral vector further comprises a MA, NC, or VPR viral gene.

23. The viral vector of claim 22, wherein the MHC polypeptide, SVGmu, mutant VSV-G, and/or detection gene expressed from the viral vector is in a fusion protein with the viral gene.

24. The viral vector of any one of claims 20-23, wherein the viral vector comprises a lentiviral, herpes simplex viral (HSV), or vaccinia viral vector.

25. A host cell comprising the chimeric protein of any one of claims 3-17, the nucleic acid of claim 19, or the viral vector of any one of claims 20-24.

26. The host cell of claim 25, wherein the host cell comprises a viral packaging cell.

27. A host cell comprising all or a portion of a MHC polypeptide, wherein the host cell is a viral packaging cell line.

28. A host cell of claim 27, wherein the MHC polypeptide is non-covalently linked to the host cell through one or more adaptor molecules.

29. A host cell comprising one or more nucleic acids encoding all or a portion of a MHC polypeptide, wherein the host cell is a viral packaging cell line.

30. The host cell of any one of claims 26-29, wherein the MHC polypeptide comprises a class I or class II MHC.

31. The host cell of any one of claims 26-30, further comprising an antigen or fragment thereof.

32. The host cell of any one of claims 26-31, further comprising a nucleic acid encoding an antigen or fragment thereof.

33. The host cell of claim 31 or 32, wherein the antigen or fragment thereof comprises a peptide.

34. The host cell of claim 33, wherein the peptide is 13-25 amino acids in length.

35. The host cell of claim 33 or 34, wherein the cell comprises a fusion protein or a nucleic acid encoding a fusion protein, wherein the fusion protein comprises the peptide, class I heavy chain, and β2-microglobulin light chain.

36. The host cell of claim 35, wherein the fusion protein comprises a single chain trimer (SCT).

37. The host cell of any one of claims 26-36, wherein the host cell comprises a viral envelope protein with disrupted native receptor binding that retains fusion activity.

38. The host cell of claim 37, wherein the host cell comprises mutant VSV-G or SVGmu.

39. The host cell of any one of claims 26-38, wherein the cell further comprises a nucleic acid encoding a detection gene.

40. The host cell of claim 39, wherein the detection gene encodes for a fusion protein of a detection gene and a viral protein.

41. The host cell of claim 40, wherein the viral protein comprises MA, NC, or VPR.

42. The host cell of any one of claims 26-41, wherein the cell further comprises a nucleic acid encoding a selective advantage gene.

43. The host cell of claim 42, wherein the selective advantage gene comprises a pro-survival gene, a pro-proliferative gene, or an antibiotic resistance gene, or combinations thereof.

44. The host cell of any one of claims 39-43, wherein the detection gene and/or selective advantage gene are encoded in the viral genome on a viral vector.

45. The host cell of claim 44, wherein the nucleic acids comprising the detection gene and/or selective advantage gene are flanked at the 3′ and/or 5′ ends by long terminal repeats (LTRs) that facilitate the insertion of the detection and/or selective advantage genes into the genome of an infected cell.

46. The host cell of any one of claims 25-45 further comprising one or more proteins or nucleic acids that encode for proteins that facilitate viral production in the cells, wherein at least one of the proteins comprises an envelope protein that is T cell non-tropic and fusogenic.

47. The host cell of any one of claims 25-46, wherein one or more of the nucleic acids are integrated into the genome of the host cell.

48. The host cell of any one of claims 25-46, wherein one or more of the nucleic acids are transiently expressed in the host cell.

49. The host cell of any one of claims 26-48, wherein the host cell is deficient for HLA.

50. The host cell of claim 49, wherein the host cell comprises a HLA gene disruption.

51. The host cell of any one of claims 26-50, wherein the host cell comprises or further comprises a peptide library comprising a plurality of nucleic acids, wherein each nucleic acid encodes for one peptide and wherein the library encodes for 2-1000 different peptides.

52. The host cell of claim 51, wherein each nucleic acid further comprises a barcode.

53. The host cell of claim 51, wherein the barcode is flanked at the 3′ and/or 5′ ends by long terminal repeats (LTRs) that facilitate the insertion of the barcode into the genome of an infected cell.

54. The host cell of any one of claims 26-53, wherein the MHC polypeptide is not flanked at the 3′ and 5′ ends by LTRs.

55. The host cell of any one of claims 26-54, wherein an endogenous MHC gene is mutated.

56. The host cell of claim 55, wherein the endogenous MHC gene is mutated to reduce or eliminate protein expression, protein activity, or protein cell membrane localization.

57. A method comprising incubating the host cell of any one of claims 26-56 under conditions suitable for the production of viral particles and isolating viral particles.

58. The method of claim 57, wherein the method further comprises contacting the viral particles with one or more peptides that bind to the MHC polypeptide.

59. A viral particle produced by the host cell of any one of claims 26-56 or by the method of claim 57 or 58.

60. A viral particle comprising all or a portion of a MHC polypeptide linked to the surface of the viral envelope.

61. The viral particle of claim 60, wherein the MHC polypeptide is non-covalently linked to the envelope through one or more adaptor molecules.

62. The viral particle of claim 61, wherein the MHC polypeptide is non-covalently linked to the envelope through biotin and a biotin-binding peptide.

63. The viral particle of claim 61 or 62, wherein a peptide is covalently or non-covalently linked to the MHC polypeptide.

64. The viral particle of claim 60, wherein the MHC polypeptide is linked to the envelope through a transmembrane embedded within the membrane of the envelope.

65. The viral particle of claim 60, wherein the MHC polypeptide comprises a chimeric protein comprising a MHC polypeptide and a viral protein or a fragment thereof.

66. The viral particle of claim 65, wherein the MHC polypeptide is at the C-terminal or N-terminal end of the viral protein or viral protein fragment.

67. The viral particle of claim 65 or 66, wherein the viral protein or fragment comprises a protein expressed from the viral gag or env genes, or a fragment thereof.

68. The viral particle of any one of claims 65-67, wherein the viral protein or fragment comprises a viral envelope protein and wherein the envelope protein is fusogenic and non-tropic for T cells.

69. The viral particle of claim 68, wherein the viral protein comprises a viral envelope protein and wherein the envelope protein is fusogenic and non-tropic for human T cells.

70. The viral particle of claim 68 or 69, wherein the envelope protein comprises a viral glycoprotein.

71. The viral particle of claim 70, wherein the viral glycoprotein comprises SVGmu.

72. The viral particle of any one of claims 65-71, wherein the viral protein comprises an ecotropic envelope protein or a fragment thereof.

73. The viral particle of any one of claims 65-72, wherein the viral protein comprises a transmembrane region.

74. The viral particle of any one of claims 60-64, wherein the MHC polypeptide comprises a chimeric protein comprising a MHC polypeptide and a transmembrane domain.

75. The viral particle of claim 74, wherein the transmembrane domain comprises a transmembrane domain from a tetraspanin protein.

76. The viral particle of any one of claims 60-75, wherein the MHC polypeptide comprises a polypeptide from a class I or class II MHC.

77. The viral particle of any one of claims 60-77, wherein a peptide is linked to the MHC polypeptide.

78. The viral particle of claim 77, wherein the peptide is 13-25 amino acids in length.

79. The viral particle of claim 77 or 78, wherein the peptide is linked to the MHC polypeptide through non-covalent interactions.

80. The viral particle of claim 77 or 78, wherein the peptide is linked to the MHC polypeptide through a covalent bond and wherein the covalent bond comprises a peptide bond.

81. The viral particle of claim 80, wherein the peptide linked to the MHC polypeptide comprises a fusion protein comprising a single chain trimer (SCT).

82. The viral particle of any one of claims 60-81, wherein the virus further comprises a nucleic acid encoding a detection gene.

83. The viral particle of any one of claims 60-82, wherein the virus further comprises a nucleic acid encoding a selective advantage gene.

84. The viral particle of claim 83, wherein the selective advantage gene comprises a pro-survival gene, a pro-proliferative gene, or an antibiotic resistance gene, or combinations thereof.

85. The viral particle of any one of claims 82-84, wherein the nucleic acids comprising the detection gene and/or selective advantage gene are flanked at the 3′ and/or 5′ ends by long terminal repeats (LTRs) that facilitate the insertion of the detection and/or selective advantage genes into the genome of an infected cell.

86. A virus comprising a plurality of viral particles according to any one of claims 59-85

87. A virus comprising one or more viral particles according to any one of claim 57 or 77-86, wherein each viral particle comprises a barcode that is unique to the peptide.

88. The virus of claim 87, wherein the barcode is flanked at the 3′ and/or 5′ ends by long terminal repeats (LTRs) that facilitate the insertion of the barcode into the genome of an infected cell.

89. A method for labeling and/or selecting antigen-specific T cells, the method comprising infecting a population of T cells with the virus of any one of claims 86-88, wherein T cells specific for the peptide-MHC polypeptide on the surface of the virus are infected with the virus.

90. The method of claim 89, wherein the T cells comprise PBMCs or TIL.

91. The method of claim 89 or 90, wherein the method further comprises selecting for the antigen-specific T cells.

92. The method of any one of claims 89-91, wherein the method further comprises detecting cells expressing the detection gene and isolating and/or counting the detected cells.

93. The method of claim 92, wherein isolating and/or counting the detected cells comprises flow cytometry, microscopy, or gel electrophoresis.

94. The method of claim 93, wherein the method further comprises sequencing all or part of the genome of the isolated cells.
